(19) United States
(12) Patent Application Publication
(10) Pub. No.: US 2019/0381144 A1
(43) Pub. Date: Dec. 19, 2019

(54) Prolactin Delivering Nerve Wrap

(71) Applicant: Michael Friel, Mandeville, LA (US)

(72) Inventor: Michael Friel, Mandeville, LA (US)

(21) Appl. No.: 16/012,359

(22) Filed: Jun. 19, 2018

(51) Int. Cl. A61K 38/22; A61L 27/18; A61L 27/38

(52) U.S. Cl. CPC A61K 38/2257; A61B 17/1128; A61L 27/383; A61L 27/18

(57) ABSTRACT

A tissue graft construct, materials and methods for repairing an injured nerve in a patient. The tissue graft construct includes a band of biocompatible matrix and the hormone prolactin. The graft can be formed into a conduit and used to deliver the hormone prolactin directly at the site of nerve injury to promote healing.

### CROSS-REFERENCE TO RELATED APPLICATION

Not applicable.

### STATEMENT REGARDING FEDERALLY SPONSORED RESEARCH OR DEVELOPMENT

Not Applicable.

### BACKGROUND

### Field of the Inventions

The field of the invention relates to a tissue graft that delivers the hormone prolactin to the site of an injured nerve for promoting the repair of nerve tissue.

### Background Information

The peripheral nervous system (PNS) consists of all neurons that exist outside the brain and spinal cord. This includes long nerve fibers containing bundles of axons as well as ganglia made of neural cell bodies. The peripheral nervous system is a network of forty three pairs of motor and sensory nerves that connect the central nervous system (CNS) made of the brain and spinal cord to various parts of the body and receives input from the external environment as well.

Unlike the CNS where the brain is protected by the skull and the spinal cord is protected by the vertebrae, the PNS is fragile and can be damaged easily through injury or trauma, and the PNS is also susceptible to disease resulting in impaired function. A peripheral nerve injury typically occurs when a nerve is compressed, crushed or partially or fully severed and proper communication between the PNS and CNS is lost A peripheral nerve injury is frequently located in the upper extremities and associated with a sub-optimal recovery of arm and hand function, loss of the capacity to move fingers and other joints, and sometimes a loss of sensation in the entire limb often resulting in a significant reduction in the quality of life.

Many methods have been utilized to promote or facilitate nerve regeneration in peripheral nerve injuries and nerve repair solutions are extensively studied. Some current, methods that are known make use of a nerve wrap or conduit to help contain or guide nerve sprouts to a required destination to reestablish connection of a severed nerve. The nerve wrap serves to protect injured nerves and to reinforce reconstruction while preventing soft tissue attachments.

Despite the extensive study and use of the systems and methods for nerve repair discussed above, consistent reproducible nerve reconstruction outcomes remain illusive. To address these and other problems, associated with current systems and methods for nerve repair, there is a need for nerve repair conduits that allow for the delivery of molecular and cellular-based therapy to optimize rehabilitative potential.

### SUMMARY OF THE INVENTION

Systems and methods for the use of a prolactin delivering nerve wrap are disclosed. In one aspect, the prolactin delivering nerve wrap comprises a tissue graft construct for promoting the repair of damaged or diseased neurological related tissue with the nerve wrap comprising a band of biocompatible matrix and prolactin hormone.

In another aspect, the prolactin hormone is embedded in the biocompatible matrix and can be configured to be a time released hormone. Alternatively, the prolactin hormone can be applied to the biocompatible matrix as a coating.

In another aspect, the band of biocompatible matrix is configured to be formed into a biodegradable tube having an interior portion surrounding the damaged or diseased neurological related tissues where the prolactin hormone is provided on the interior portion of the biodegradable tube.

These and other features, aspects, and embodiments of the inventions are described below in the section entitled “Detailed Description of the Preferred Embodiments.”

### BRIEF DESCRIPTION OF THE DRAWINGS

Features, aspects, and embodiments of the inventions are described in conjunction with the attached drawings, in which:

FIG. 1 illustrates a perspective view of a tubular graft prosthesis structure in accordance with one example embodiment of the invention.

### DETAILED DESCRIPTION OF THE PREFERRED EMBODIMENTS

The peripheral nervous system (PNS) consists of all neurons that exist outside the brain and spinal cord. This includes long nerve fibers containing bundles of axons as well as ganglia made of neural cell bodies. The peripheral nervous system is a network of forty three pairs of motor and sensory nerves that connect the central nervous system (CNS) made of the brain and spinal cord to various parts of the body and receives input from the external environment as well.

Functionally, the PNS is divided into sensory (afferent) and motor (efferent) nerves, depending on whether they bring information to the CNS from sensory receptors or carry instructions towards muscles, organs or other effectors. Motor nerves can be further classified as somatic or autonomic nerves, depending on whether the motor activity is under voluntary conscious control

The primary function of the PNS is to connect the brain and spinal cord to the rest of the body and the external environment. This is accomplished through nerves that carry information from sensory receptors in the eyes, ears, skin, nose and tongue, as well as stretch receptors and nociceptors muscles, glands and other internal organs. When the CNS integrates these varied signals, and formulates a response, motor nerves of the PNS innervate effector organs and mediate the contraction or relaxation of skeletal, smooth or cardiac muscle.

Thus, the PNS regulates internal homeostasis through the autonomic nervous system, modulating respiration, heart rate, blood pressure, digestion reproduction and immune responses. It can increase or decrease the strength of muscle contractility across the body, whether it is sphincters in the digestive and excretory systems, cardiac muscles in the heart or skeletal muscles for movement. It is necessary for all voluntary action, balance and maintenance of posture and for the release of secretions from most exocrine glands. The PNS innervates the muscles surrounding sense organs, so it is involved in chewing, swallowing, biting and speaking. At the same time, it mediates the response of the body to noxious stimuli, quickly removing the body from the injurious stimulus, whether it is extremes in temperature, pH, or pressure, as well as stretching and compressing forces.

The PNS is made of nerves, ganglia and plexuses. A nerve contains the axons of multiple neurons bound together by connective tissue. The axon itself is often myelinated, containing a phospholipid secreted by a glial cell called the Schwann cell. The thin covering of Schwann cell cytoplasm forms the innermost layer protecting an axon and is called the neurilemma or neurolemma. Blood capillaries and other connective tissue around the neurilemma form the endoneurium. When multiple axons are bundled together to form structures called fascicles, a fibrous tissue called the perineurium holds them together. Finally, the whole nerve containing numerous axon bundles is encased in fibrous epineurium. The cell bodies or soma of these neurons also cluster together and are covered by the epineurium to form ganglia that look like swellings on the nerve fiber. In the autonomic nervous system, these ganglia become the sites for synaptic transmission between two neurons. Branching networks of intersecting spinal and autonomic nerves form structures called plexuses that have both sensory and motor functions and serve a particular region of the body.

Unlike the CNS where the brain is protected by the skull and the spinal cord is protected by the vertebrae, the PNS is fragile and can be damaged easily through injury or trauma, and the PNS is also susceptible to disease resulting in impaired function. A peripheral nerve injury typically occurs when a nerve is compressed, crushed or partially or fully severed and proper communication between the PNS and CNS is lost. A peripheral nerve injury is frequently located in the upper extremities and associated with a sub-optimal recovery of arm and hand function, loss of the capacity to move fingers and other joints, and sometimes a loss of sensation in the entire limb often resulting in a significant reduction in the quality of life.

Many methods have been utilized to promote or facilitate nerve regeneration in peripheral nerve injuries and nerve repair solutions are extensively studied. For purposes of discussion of the preferred embodiments herein, an extensive discussion of previously attempted methods of repair is not necessary. Instead, the discussion shall be focused on the following current methods that are known and used to help contain or guide nerve sprouts to a required destination to reestablish connection of a severed nerve.

When a peripheral nerve is cut, it is separated into two parts, the proximal part and the distal part. The proximal side, of the nerve is closest to the spinal cord and is still in communication with the CNS. The distal side of the nerve is farther away from the spinal cords and has lost communication with the central nervous system. Once transected, the axons in the distal nerve begin to break down and the body initiates a process to clear the resulting cellular debris. This process is known as Wallerian degeneration and results in mainly hollow tubes in the nerve where axons used to be. Specialized cells called Schwann cells proliferate from both the proximal and distal nerve stumps to support regeneration. The proximal end of the nerve begins to sprout toward the distal nerve. Studies have shown that the nerve fiber advances at about 1 mm per day and may eventually reach its target tissue where it can once again provide sensation or movement. However, without some form of guidance, these nerve sprouts often die back into the proximal nerve or potentially form a neuroma. Retrieved from https://www.axogeninc.com/for-patients/how-peripheral-nerves-repair [online][retrieved Jun. 8, 2018]

In some cases, a partially or fully severed nerve can be reattached in a tension free manner wherein the epineurium surrounding the ends of the injured nerve can be sutured together. The primary goal in repairing the nerve is to save and reestablish the insulating cover so that new fibers can regrow and the nerve can begin to function again. During the healing process, however, the injured nerve remains susceptible to reinjury.

To assist in the healing process, one currently available commercial product called the AxoGuard® Nerve Protector which is produced by Axogen, Inc. (AXGN), is utilized during the surgical repair to help protect the injured nerve during the healing process. The AxoGuard® Nerve Protector's product insert contains patent markings identifying U.S. Pat. Nos. 6,241,981 and 6,206,931, the entire disclosures of which, except for any definitions, disclaimers, disavowals, and inconsistencies, are incorporated herein by reference.

The AxoGuard® Nerve Protector is currently the only porcine submucosa extracellular (EMC) matrix surgical implant used to protect injured nerves and to reinforce reconstruction while preventing soft tissue attachments. Designed to protect and isolate, the AxoGuard® multi-laminar ECM separates and protects the nerve from the surrounding tissues during the healing process. The patient's own cells incorporate into the minimally processed extracellular matrix to remodel and form a tissue similar to the nerve epinerium. AxoGuard® Nerve Protector is provided sterile and in a variety of sizes to meet the surgeon's and anatomical needs. The AxoGuard® Nerve Protector can be used to protect injured nerves up to 40 mm; minimize the risk of entrapment in compressed nerves; protect partially severed nerves; and reinforce a coaptation site. The AxoGuard® Nerve Protector also has the following key advantages: minimizes the potential for soft tissue attachments and nerve entrapment by physically isolating the nerve during the healing process; preserves the native architecture of the extracellular matrix; allows nerve gliding; strong and flexible while still being easy to suture; stored at room temperature with an 18 month shelf life. Retrieved from https://www.axogeninc.com/products/4144/axoguard-nerve-protector [on line][retrieved Jun. 8, 2018] There are other commercially available nerve wrap products available, with the leading competitor being the Integra® NeuraGen® Nerve Guide produced by Integra Life Sciences.

When tension free primary repair is not possible, a suitable alternative repair to the injured nerve must be pursued in order to bridge the gap between the proximal and distal end of the injured nerve. The surgical technique employed in these alternatives is similar, whether it be a nerve graft, nerve transfer, or conduit.

Autogenous nerve grafts have long been considered the gold standard for repair of irreducible nerve grafts. Autogenous grafts act as immunogenically inert scaffolds, providing appropriate neurotrophic factors and viable Schwann cells for axonal regeneration. The choice of autogenous graft is dependent on several factors: the size of the nerve gap, location of proposed nerve repair, and associated donor-site morbidity. Although the sural nerve is the most commonly used autograft, there are many other suitable nerves that can be used as interposition grafts including: the medial and lateral cutaneous nerves of the forearm, dorsal cutaneous branch of the ulnar nerve, superficial and deep peroneal nerves, intercostal nerves, and the posterior and lateral cutaneous nerves of the thigh. Once the autogenous nerve graft has been sutured in place, axon regeneration begins and within weeks, the regenerating axons that have found a tube to guide it are close to bridging the entire length of the nerve gap. Over time these axons regenerate across the entire length of the graft then continue on to the target site (e.g., muscle or skin). Over the next several months, these axons continue to mature and get thicker and remyelinate resulting in a new nerve segment that, depending on the injury and nerve being repaired, may be thinner than the recipient's own nerve yet is functional.

A nerve transfer is a surgical technique wherein the surgeon selects a redundant nerve—one that serves the same function as another nerve in the body—and connects it to a more important but damaged nerve that is not working. During this procedure, the surgeon leaves intact the donor nerve's connection with the spinal cord and mobilizes a distal portion of donor nerve—a section long enough to reach the injured nerve. The mobilized section of donor nerve is moved from its original location to the location of the injured nerve. The end of the donor nerve is coapted to the injured nerve. Once the nerve fibers are connected, the nerve with a healthy connection to the spinal cord can grow down through the, conduit provided by the injured nerve. The nerve protector can also be used in nerve transfers. A common transfer is the Anterior interosseous nerve to ulnar nerve at the wrist for high ulnar nerve injuries.

In another approach to bridge the gap of an injured nerve, nerve allografts make use of cadaveric nerve segments. The processing of those nerves is proprietary and distributed as the Avance® Nerve Graft by Axogen, Inc. The Avance® Nerve Graft is an off-the-shelf processed human nerve allograft intended for the surgical repair of peripheral nerve discontinuities. Through a proprietary cleansing process for recovered human peripheral nerve tissue, the graft preserves the essential inherent structure of the extra cellular matrix while cleansing away cellular and noncellular debris. The Avance® Nerve Graft provides the following advantages: three dimensional scaffold for bridging a nerve gap; decellularized and cleansed extracellular matrix that remodels into patient's own tissue; no donor nerve surgery; and therefore no comorbidities associated with an additional surgical site; available in a variety of lengths and diameters to meet a range of gap lengths and anatomical needs; and supplied sterile with three years shelf life when kept frozen. Retrieved from https://www.axogeninc.com/products/4134/avance-nerve-graft [online][retrieved Jun. 8, 2018] Once implanted, the Avance® Nerve Graft provides many open tubes that provide a physical scaffold for axon and cell in growth. Over time, these axons regenerate across the entire length of the graft and continue on to the target site (e.g., muscle or skin). Over the next several months, these axons continue to mature and get thicker and remyelinate resulting in a new nerve segment that, depending on the injury and nerve being repaired, is closely sized to the recipient's own nerve and is functional. Retrieved from https://www.axogeninc.com/for-patents/how-peripheral-nerves-repair [online][retrieved Jun. 8, 2018]

In each of the above referenced procedures of autogenous nerve grafts, nerve transfers, and nerve allografts, a nerve wrap such as the AxoGuard® Nerve Protector can be used to wrap around nerve anastomosis sites to minimize the risk of entrapment and protect and reinforce the coaptation site. Further, the nerve protector can provide a favorable environment for nerve regeneration.

Finally, in another approaching to bridge the gap in an injured nerve, a considerable amount of research has been devoted to the development of a viable synthetic or biological nerve conduit and currently several commercially available options exist. One leading, commercial nerve conduit is the AxoGuard® Nerve Connector distributed by AxoGen, Inc. The AxoGuard® Nerve Connector is the only porcine submucosa extracellular matrix (“ECM) coaptation aid for tensionless repair of transected or severed peripheral nerves. While allowing for the close approximation of severed nerves, AxoGuard® Nerve Connector aligns and connects severed nerve ends with less than a 5 mm gap. The AxoGuard® Nerve Connector ECM material allows the body's natural healing process to repair the nerve by isolating and protecting it during the healing process. The patient's own cells incorporate into the extracellular matrix to remodel and form a tissue similar to nerve epineurium. AxoGuard® Nerve Connector can be used to relieve tension, at the coaptation site of severed nerves, aid coaptation in direct repair, grafting, or cable grafting repairs, and reinforce the coaptation site. Retrieved from https://www.axogeninc.com/products/4124/axoguard-nerve-connector [online][retrieved Jun. 8, 2018]

Within hours following implantation of a conduit, fluid containing inflammatory cells and secreted trophic factors fills the conduit. Within days, a fibrin cable forms inside the tube. Regeneration cannot successfully occur within a conduit, without the formation of this fibrin matrix, which serves an essential function as a physical bridge across the nerve gap and provides contact guidance for cells. Notably the thickness and quality of the cable, which effectively provides the total available regeneration area, is inversely related to the size of the conduit and often does not fill the entire volume of the tube or does not even form at all if the gap length is too long. Over the next several weeks following implantation, Shwann cells infiltrate from both stumps and grow along the fibrin cable. Some of the Shwann cells are able to line up and form aligned tubes making a structure that can physically guide regenerating axons across the nerve gap. Axon regeneration is centralized to where the fibrin cable forms. Some of these Shwann cells tubes remain empty and any regenerating axons that do not locate a Shwann cells tube to guide it get pruned back and die. Over time, axons that are able to find and regenerate within a Shwann cell tube grow across the entire length of the conduit and continue on to the target site e.g. muscle or skin. Timing for the stability of conduits is crucial. Conduits need to remain stable until regeneration occurs across the nerve gap but once regeneration has occurred, the conduit is useless and its continued presence is only a potential hazard which is why the majority of nerve conduits are composed of not permanent, materials that, degrade or resorb over time. Depending on the size of the nerve discontinuity and on the type of injury, adequate nerve repair can be achieved but often the resulting new nerve segment is thinner and its function is highly variable. Retrieved from https://www.axogeninc.com/axogen-resources-science-of-nerve-repair[online][retrieved Jun. 8, 2018]

Despite the extensive study and use of the systems and methods for nerve repair discussed above, consistent reproducible nerve reconstruction outcomes remain illusive. As published in Plastic and Reconstructive Surgery, biological approaches to nerve reconstruction are needed to improve nerve reconstruction. Kubiak C A, Kung T A, et. Al State-of-the-Art Techniques in Treating Peripheral Nerve Injuries. Plastic an Reconstructive Surgery March 2018:141(13). p-702-710). The “third generation” of nerve repair conduits relate to the development of conduits that allow for the delivery of molecular and cellular-based therapy to optimize rehabilitative potential. Nerve conduits seeded with stem cell and Schwann cells have demonstrated better function outcomes compared to empty conduits. Zhang B G, Quigley A F et al. Recent advances in nerve tissue engineering. Int J Artif Organs 2014; 37:277-291 Further, controlled release of regenerative neurotrophicc factors from conduits have also been shown to be beneficial for axonal, growth. Cui Q. Actions of Neurotrophic Factors and their signaling pathways in neuronal survival and axonal regeneration. Mol Neurabiol, 2006, 33:155-179

Prolactin is a 23 kDa polypeptide hormone mainly secreted by the lactotrophic cells of the anterior pituitary gland, even though other cell types can also release this hormone, including immune cells, adipocytes, mammary and epithelial cells. As discussed further below, prolactin has been found to promote remyelination and neurogenesis.

Multiple sclerosis (MS) is a chronic, inflammatory disorder of the CNS. Neurological symptoms include, sensory disturbances, limb weakness, fatigue, and sexual and bladder dysfunctions. MS is believed to be the result of a cell-driven autoimmune response against myelin in the CNS. Inflammation triggers demyelination and axonal injury, resulting in defective propagation of action potentials through the internodes of nerves (loss of saltatory conduction) and neurological symptoms. The main histological feature of acute MS is represented by the formation of plaques in multiple sites with CNS white matter. These areas are infiltrated by peripheral immune cells, such as macrophages, T cells, B lymphocytes and plasma cells, and display evidence of myelin loss and axonal injury.

It is well documented that women affected by MS experience a substantial reduction of disease activity during the third trimester of pregnancy, and an increased rate of disease activity in the trimester after delivery. These observations suggested a hormonal influence on MS pathogenesis. In a collaboration between the laboratories of Drs. Samuel Weiss and V. Wee Yong of the Hotchkiss Brain Institute, their study was the first to determine that prolactin, which increases in the female body during pregnancy, is directly responsible for the formation of new myelin in the brains and spinal cords of pregnant mice. Further, when non-pregnant mice with MS-like lesions were injected with prolactin, their myelin was also repaired. (Costanza et al. [2016] Int J Mol Sci 17(12):2026; Gregg et al. [2007] Journal of Neuroscience 27(8):1812-1823).

Several studies have also demonstrated that prolactin can positively affect neurogenesis in several physiological and pathological conditions. Prolactin is important for pregnancy-stimulated neurogenesis of the female adult brain, a process that probably supports maternal adaptation to offspring (Costanza et al. [2016] Int J Mol Sci 17(12):2026; Shingo et al. [2003] Science 299(5603):117-20). PRLR has been detected on the choroid plexus and on the dorsolateral corner of the subventricular zone (SVZ) one of the neurogenic areas of the adult forebrain (Costanza et al. [2016] Int J Mol Sci 17(12):2026; Shingo et al. [2003] Science 299(5603): 117-20). When adult neurospheres are cultured in vitro in the presence of prolactin, the number of differentiated neurons is doubled. (Costanza et al. [2016] Int J Mol Sci 17(12):2026; Shingo et al. [2003] Science 299(5603):117-20). Prolactin-deficient mice display learning and memory deficits which are rescued by administration of prolactin into the hippocampus. (Costanza et al. [2016] Int J Mol Sci 17(12):2026; Walker et al. [2012] PLoS One 7(9):e44371). The impact of prolactin on neurogenesis has also been evaluated during chronic exposure to stress, a condition that results in the diminishment of hippocampal neurogenesis. It was found that repeated peripheral administration of prolactin to adult mice subjected to chronic stress—four hours of daily immobilization for twenty one days—can rescue adult hippocampal neurogenesis. (Costanza et al. [2016] Int J Mol Sci 17(12):2026; Tomer et al. [2009] J Neurosci. 29(6):1826-33). Prolactin treatment during stress conditions induces a higher percentage of newborn neurons if compared to vehicle treated mice. (Costanza et al. [2016] Int J Mol Sci 17(12):2026; Tomer et al. [2009] J Neurosci. 29(6):1826-33).

The actions of prolactin in the CNS are not limited to adult neurogenesis. PRL has been indicated as a promising therapeutic agent for spinal muscular atrophy (SMA), a neurodegenerative disorder characterized by the loss of motoneurons and progressive muscular atrophy (Costanza et al. [2016] Int J Mol Sci 17(12):2026; Farooq et al. [2011] J Clin Invest. 121(8):3042-50). Systemic administration of prolactin in a mouse model of severe SMA promotes a drastic improvement of motor functions, associated with a slowdown of weight loss and enhanced survival. (Costanza et al. [2016] Int J Mol Sci 17(12):2026; Farooq et al. [2011] J Clin Invest. 121(8):3042-50).

Oligodendrocytes are also positively affected by prolactin stimulation. Gregg and coworkers have shown that murine pregnancy promotes the increase of oligodendrocyte precursor cells (OPCs) and myelinated axons in the female adult brain. (Costanza et al. [2016] Int J Mol Sci 17(12):2026; Gregg et al. [2007] Journal of Neuroscience 27(8):1812-1823). Pregnant mice also display enhanced remyelinating capacity following acute demyelinating injury triggered by the injection of the detergent lysolecithin in the spinal cord. This enhanced white matter plasticity is significantly impaired in pregnant Prlr^{+/-} mice. Moreover, prolantin administration supports myelin repair in virgin females receiving lysolecithin lesions (Costanza et al. [2016] Int J Mol Sci 7(12):2026; Gregg et al. [2007] Journal of Neuroscience 27(8):1812-1823).

The applicant is not aware of any published data for management of a traumatic injury to a peripheral nerve in the setting of a pregnant patient. However, as discussed above, in the central nervous system, studies have shown that multiple sclerosis has gone into remission during pregnancy, suggesting a hormonal connection to nerve regeneration. This has been attributed to the hormone prolactin, which some studies have reported as being endowed with important neuroprotective and remyelinating properties

The named inventor of the current application is a medical doctor. In the applicant's medical practice, he encountered the case of a nine week pregnant woman who sustained a deep laceration to her volar distal forearm. Several flexor tendons as well as the ulnar nerve and artery were transected. She underwent operative exploration under axillary nerve block. After debridement, there was a 20 mm gap in the ulnar nerve. This was repaired using a cadaveric allograft followed by a nerve wrap. The tendons were repaired in the usual fashion.

She recovered uneventfully and participated well with occupational therapy. She went on to deliver a healthy baby. At 10 months post-op, she had regained full sensation and M4 motor function. She has recovered full use of her hand and has returned to work as a typist at a call center for a large international telecommunications company. Applicant is not aware of any other published cases of such a remarkable recovery after complete ulnar nerve transection at the level of the wrist in the setting of pregnancy.

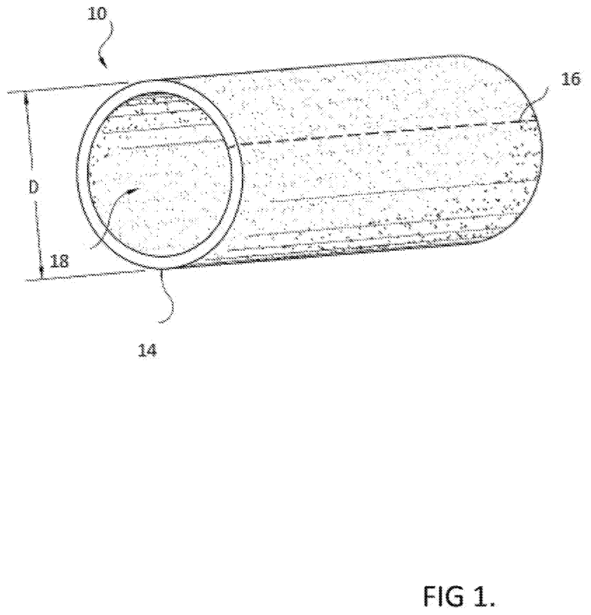

Turning now to FIG. 1, one exemplary embodiment of a tissue graft 10 is illustrated for use in the repair of a peripheral nerve tissue. A generally tubular graft prosthesis structure 12 is formed with or includes a collagen-based matrix 14, the diameter “D” of which approximates that of the recipient peripheral nerve tissue. In one embodiment, this may be accomplished by manipulating a tubular segment or sheet of the collagen-based matrix 14 to define a cylinder having a diameter “D” approximately the same as that of the recipient nerve tissue and then suturing, bonding or otherwise securing the longitudinal seam 16 to form an appropriately-dimensioned tubular graft having a lumen 18 that encompasses the damaged nerve tissue or provides a conduit between the severed ends of an inured nerve. Systems and methods for producing such a tissue graft are known and described in U.S. Pat. Nos. 6,241,981 and 6,206,931, the entire disclosures of which, except for any definitions, disclaimers, disavowals, and inconsistencies, are incorporated herein by reference.

In another embodiment, the collagen-based matrix 14 further includes a preparation of synthetic prolactin hormone embedded in the collagen-based matrix 14. Typically, the synthetic prolactin, which is readily available from commercial suppliers, is provided in the form of a powder that is reconstituted with liquid. In another embodiment, the synthetic prolactin, hormone is applied as a coating to the interior portion of the lumen 18 such that the prolactin is in close proximity to the site in need of nerve tissue repair. The synthetic prolactin hormone can be provided in a time-released formulation such that the prolactin is delivered to the site of nerve repair over an extended period of time to aid in the repair of the nerve tissue.

All patent applications, patents, and printed publications cited herein are incorporated herein by reference in the entireties, except for any definitions, subject matter disclaimers or disavowals, and except to the extent that the incorporated material is inconsistent with the express disclosure herein, in which case the language in this disclosure controls.

While certain embodiments of the inventions have been described above, it will be understood that the embodiments described are by way of example only. Accordingly, the inventions should not be limited based on the described embodiments.

1.-10. (canceled)

11. A method for inducing the formation of neurological related tissue at a site in need of neurological related tissue growth, the method comprising surgically implanting a tissue graft to couple the tissue graft to a peripheral nerve, wherein the tissue graft comprises a band of biocompatible matrix that provides a timed release of prolactin hormone in proximity to the site in need of neurological related tissue growth.

12. (canceled)

13. The method of claim 11, wherein the band of biocompatible matrix is formed into a biodegradable tube.

14. The method of claim 13, wherein the band of biocompatible matrix is formed into a biodegradable tube having an interior portion surrounding a portion of the peripheral nerve.

15. The method of claim 14, wherein the prolactin hormone is provided on the interior portion of the biodegradable tube.

16. A method for inducing the formation of neurological related tissue at a peripheral nerve site, the method comprising surgically implanting a tissue graft to couple the tissue graft to a peripheral nerve, wherein the tissue graft comprises a band of biocompatible matrix and prolactin hormone.

17. The method of claim 16, wherein the prolactin hormone is embedded in the biocompatible matrix.

18. The method of claim 17, wherein the prolactin hormone is configured to be a time released hormone.

19. The method of claim 16, wherein the prolactin hormone is applied to the band of the biocompatible matrix as a coating.

20. The method of claim 19, wherein the prolactin hormone is configured to be a time released hormone.

21. The method of claim 20, wherein the band of biocompatible matrix comprises a purified, collagen-based matrix structure removed from a submucosa tissue source.

22. The method of claim 21, wherein the band of biocompatible matrix is formed into a biodegradable tube.

23. The method of claim 22, wherein the band of biocompatible matrix is formed into a biodegradable tube having an interior portion surrounding a portion of the peripheral nerve.

24. The method of claim 23, wherein the prolactin hormone is provided on the interior portion of the biodegradable tube.
